(19) United States
(12) Patent Application Publication
(10) Pub. No.: US 2026/0326108 A1
(43) Pub. Date: Oct. 22, 2026

(54) SET COMPRISING A MICROPLATE, A LID, AND A DEVICE FOR RELEASING A SNAP CONNECTION BETWEEN THE MICROPLATE AND THE LID

(71) Applicant: EPPENDORF SE, Hamburg (DE)

(72) Inventor: Detlef Schwarzwald, Hamburg (DE)

(73) Assignee: EPPENDORF SE, Hamburg (DE)

(21) Appl. No.: 19/107,684

(22) PCT Filed: Aug. 15, 2023
(86) PCT No.: PCT/EP2023/072444

§ 371 Date: Feb. 28, 2025

(30) Foreign Application Priority Data
Sep. 2, 2022 (EP) 22193653.7

(51) Int. Cl. B01L 3/00

(52) U.S. Cl. CPC B01L 3/50853; B01L 2200/087; B01L 2300/041; B01L 2300/0829

(57) ABSTRACT

A microplate set includes a microplate, lid, and an unlocking device. The microplate includes a first plate including a upper side and an underside, a plurality of wells that each comprises a top that is connected to the first plate and projects downward from the underside of the first plate and defines an opening on the upper side of the first plate, and snap openings on at least two opposing lateral edges of the first plate. The lid includes a second plate configured for placement onto the upper side of the first plate, and a plurality of snap hooks projecting from an underside of the second plate on at least two opposing lateral edges of the second plate. The lid is configured to snap into the snap openings via the plurality of snap hooks. The unlocking device is configured for releasing the snap connections between the microplate and the lid.

### CROSS-REFERENCE TO RELATED APPLICATIONS

This is a U.S. National Application of PCT/EP2023/072444, filed Aug. 15, 2023, and claiming the priority and benefit, under relevant portions of 35 U.S.C. § 119, of European Patent Application 22193653.7, filed Sep. 2, 2022. The entire contents of said applications are hereby incorporated by reference.

### TECHNOLOGICAL FIELD

The following disclosure relates to a set comprising a microplate, also referred to as a microtiter plate, microtest plate, or multiwell plate, a lid for covering the microplate, and a device for releasing a snap connection between the microplate and lid.

### BACKGROUND

Microplates are used, in particular, in scientific and industrial laboratories with medical, molecular biology, or pharmaceutical applications for a wide variety of microbiological, cell biology, and immunological work processes. For example, microplates are used in PCR and in the cultivation of microorganisms or cells.

Microplates have a frame with a large number of wells for receiving sample liquid. Designs in which the wells are designed as depressions in an overall planar frame are known. In other designs, the wells are connected at the top to a plate of the frame, project downward from the underside of the plate, and comprise an opening on the upper side of the plate. The outer edge of the plate may be connected to a downwardly projecting, circumferential side wall of the frame. The side wall may project further downward from the plate than the wells (“skirted”). Furthermore, designs are known in which the side wall projects downward from the plate less far than the wells (“semi-skirted”). But there are also designs in which the plate is not connected to a side wall (“unskirted”). Known microplates with a side wall have an extension on the lower edge of the side wall by means of which said microplates can be placed on the upper side of another microplate. Microplates with large wells are also referred to as “deep-well plates”.

Microplates are standardized by means of the standards of the ANSI/SLAS for microplates, in particular with regard to the footprint and the arrangement of the wells in microplates with 96, 384, and 1,536 wells. Microplates with 96 wells are particularly common, in which the wells in eight rows and twelve columns are at a distance of 9 mm from one another.

Microplates and deep-well plates having 48 wells in six rows and eight columns are also known and are used, for example, for cell cultures. These microplates are offered with the same footprint or rather length and width as the microplates with 96 wells according to the standards of the ANSI/SLAS for microplates.

DE 100 66 431 B4 describes a microplate consisting of plastic material having a rigid lid that can be releasably attached to the upper side of the plate and at least one seal between the lid and the plate made of resilient material. In one exemplary embodiment, plug-shaped seals project from the underside of the plate and engage in openings of the wells so as to abut the inner wall of the wells in a sealing manner with sealing beads on their outer periphery when the lid has been placed on the microplate. The lid has borders which project downward from the edge of the plate and from which latching projections project inward, which latching projections can be latched into recesses in the side wall below the plate of the microplate. The borders of the lid have upwardly projecting handles which make it easier to grip the lid. By pivoting the handles, the latching between the latching projections and the recesses can be released, because the borders are pivoted as well. The microplate and the lid are unsuitable for automated plate handling, in particular due to the fact that the snap connection is released using the handles.

Proceeding from this, one of the objects of the invention is to provide a technology that allows for simple release of a snap connection between the microplate and the lid that can be carried out in an automated process.

### SUMMARY

Aspects of the following disclosure are directed to embodiments of a set including a microplate having a rectangular first plate, a large number of wells which are arranged in rows and columns and connected at the top to the first plate, project downward from the underside of the first plate, and have an opening on the upper side of the first plate, and snap openings on at least two mutually opposing lateral edges of the first plate. In some embodiments, the set further includes a lid including a second plate for placement onto the upper side of the first plate and for covering the openings of the wells and snap hooks projecting from the underside of the second plate on at least two mutually opposing lateral edges of the second plate. In some embodiments, the lid rests with the second plate on the upper side of the first plate in a covering position, covers the openings of the wells, and is snapped into the snap openings by means of the snap hooks so as to form snap connections. In some embodiments, the set includes an unlocking device for releasing the snap connections between the microplate and the lid, and having at least two upwardly projecting support projections and at least one receiving portion therebetween for placing a microplate with at least two mutually opposing edge regions of the first plate onto the support projections with engagement of the wells in the at least one receiving portion. In some embodiments, the set further includes a first sliding surfaces on inner sides of the snap hooks that face one another on mutually opposing edges of the first plate and second sliding surfaces on outer sides of the support projections that face away from one another on mutually opposing sides of the receiving portion. In some embodiments, the first sliding surfaces on inner sides of the snap hooks that face one another are inclined upward toward one another and/or the second sliding surfaces on outer sides of the support projections that face away from one another are inclined upward toward one another.

In some embodiments of the set according to the invention, the lid is placed onto the microplate from above in order to cover the openings of the wells and to protect the liquid poured into the wells from being loss or else from contamination. The snap hooks dip into the snap openings until they engage behind edge regions of the first plate that adjoin the snap openings, as a result of which snap connections are formed between the microplate and the lid. The lid can be released from the microplate again by placing the microplate with the lid snap-fitted thereto onto the unlocking device in order to release the snap connections (“unlocking device”). When the microplate with the lid snapped thereon is placed onto the unlocking device, the snap hooks are deflected outward and the snap connections undone on account of the sliding of the second sliding surfaces on the first sliding surfaces. The snap connections are released again merely by placing the microplate closed by means of the lid onto the unlocking device, and the lid can be lifted off of the microplate in a further movement.

In some embodiments, the snap connections between the microplate and lid can be easily established in the set according to the invention. Furthermore, the snap connections can be easily undone by placing the microplate together with the lid snapped thereon onto the unlocking device. Furthermore, automated handling of the lid is possible. The closing and snap-fitting of the microplate by means of the lid can take place by simply placing the lid onto the microplate using an automatic machine. The snap fit can be released by simply placing the microplate covered by the lid onto the unlocking device using an automatic machine. Manual handling is also possible.

Another advantage is that existing microplates can be used without modifying the set. The set can be used both with existing microplates and with adapted microplates and deep-well plates. The establishment of the snap connections and the release of the snap connections can be carried out automatically by means of a laboratory machine or dosing machine or manually by means of laboratory personnel.

In some embodiments of the invention, each snap connection is arranged at least in part in the two lateral edges of the first plate. As a result, the release of the snap connection by deflecting the snap hooks outward is made simpler, because the snap hooks come free by their ends entering the snap openings of a first plate.

In some embodiments, the snap openings may exclusively be formed in the lateral edges of the first plate, in particular in a microplate that has no side wall. A microplate with a side wall may also comprise the snap openings exclusively in the first plate, but the snap openings must be made large enough for the snap hooks to be deflected until they no longer engage under the first plate and can be pulled out of the snap openings.

According to another embodiment, the microplate comprises a circumferential side wall that projects downward from the outer edge of the first plate, and each snap opening is formed at least in part in the upper edge region of the side wall. Due to the fact that each snap opening is formed at least in part in the upper edge region of the side wall, the release of the snap connections by means of lateral deflection of the snap hooks is facilitated.

In a preferred embodiment, each snap opening is arranged in one part in the lateral edge region of the first plate and in another part in the upper edge region of the side wall. This embodiment exhibits the above advantages in combination. In some embodiments, the snap connections may also be formed exclusively in the side wall.

Furthermore, by forming the snap openings both on the edges of the first plate and in the side wall, it is easier to remove the microplate from the injection-molding tool during manufacture by means of injection molding.

According to another embodiment, the part of the snap openings that is formed in the lateral edge region of the first plate comprises an upper boundary surface that extends parallel to the outer edge of the first plate and that is flush with the inner side of the side wall or is offset inward relative thereto, which inner side adjoins a lower boundary surface of the part of the snap opening in the upper edge region of the side wall. This makes it easier to remove the microplate from the injection-molding tool during manufacture by injection molding.

According to another embodiment, the microplate comprises two snap openings in each case on the two long edges of the first plate and the lid comprises two snap hooks in each case on the two long edges of the second plate. This facilitates uniform, sealing abutment of the lid on the microplate.

According to another embodiment, the microplate comprises two snap openings in each case on the two short edges and the lid comprises two snap hooks in each case on the two short edges. This further promotes uniform abutment of the lid on the microplate.

According to another embodiment, the snap hooks comprise a vertical portion and an inwardly projecting horizontal portion at the lower end of the vertical portion. This produces flexible snap hooks that allow for simple snap-in into the snap openings and simple targeted release. According to another embodiment, the horizontal portion comprises a first sliding surface at the outer end on the underside.

According to another embodiment, the second plate comprises a cutout on the lateral edges next to the connection with the snap hook above the hook end of the snap hook. This simplifies removal of the lid from a molding tool during injection molding.

According to another embodiment, the microplate and/or the lid comprises at least one sealing element which circumferentially seals the edge of each opening if the lid comprises snap connections with the microplate. As a result, the sealing closure of the wells by the lid in the sealing position is improved. The sealing element is, for example, resilient planar material or a resilient material layer on the underside of the lid and seals all openings of the microplate when the lid has been snapped onto the microplate.

According to another embodiment, the lid comprises sealing elements (covers) arranged on the underside of the second plate in rows and columns, each of which sealing elements abuts an inner edge of one of the openings when the lid has been snap-fitted to the microplate. As a result, the sealing abutment of the lid on the openings of the wells in the sealing position is improved further. The sealing elements have, for example, a circular or annular base surface and circumferentially and sealingly abut the edge of the openings in the first plate and/or so as to adjoin the openings of the inner wall of the wells.

According to another embodiment, the sealing elements project downward from the underside of the lid.

According to another embodiment, each sealing element is dome-shaped (e.g. hemispherical) or conical. As a result, the sealing of the openings of the wells by the lid in the covering position is further improved.

According to another embodiment, the grid of covers is cambered. This means that the inner covers are higher than the outer covers. This is advantageous for sealing a grid of covers, since a deformed grid can thus lie flat. In other words, the regions in the center of the grid, which are deformed most or rather first, give way and only then is contact made with regions at the edge, which are deformed later.

According to another embodiment, the first plate comprises at least one further snap opening between the lateral edges, the lid comprises at least one downwardly projecting further snap hook between the two lateral edges of the second plate, wherein the further snap hook is introduced into the further snap opening and is snap-fitted therebehind on the underside of the first plate when the lid, with the second plate, abuts the upper side of the first plate of the microplate, and the further snap hook comprises at least one further first sliding surface, and/or the unlocking device comprises a further second sliding surface for releasing the snap connections, which further second sliding surface is designed to slide on the further first sliding surface when the microplate with the lid snapped on is placed on so as to undo the snap connection. As a result, the sealing abutment of the lid on the microplate in the covering position is improved further. Further snap hooks that are not arranged at the edge but rather between the wells can support the holding force and the uniform distribution of the latching effect. The snap hooks of the lid may not only be arranged at the edge but also between the wells. As a result, the covers can be pressed against the edges of the openings on the upper side of the first plate in an improved manner and can seal more reliably. For this purpose, further snap openings (through-holes) in which the further snap hooks can also latch must be present in the first plate. The principle of latching by means of snap hooks and unlatching by means of displacement using sliding surfaces on the unlocking block is also utilized here.

According to a further embodiment, the unlocking device comprises a rectangular frame, the support projections are formed by frame strips of the frame, and the receiving portion is formed by the inner sides of the frame. This allows for a structurally particularly simple design.

According to another embodiment, the set comprises at least one of the following features: (i) the microplate is manufactured from a single or from multiple plastics materials, wherein the frame of the microplate optionally consists of a different plastics material than the wells; (ii) the lid is manufactured from a single or from multiple plastics materials, wherein the second frame optionally consists of a different plastics material than the seal; and (iii) the unlocking device consists of a single or of multiple plastics materials, of metal, or of a combination of plastics material or metal.

According to another embodiment, the microplate and/or the lid and/or the unlocking device is injection-molded. According to another embodiment, the microplate and/or the lid is manufactured in a single-component or multi-component injection-molding process.

According to another embodiment, the microplate and/or the lid and/or the unlocking device is vacuum-formed and/or is composed of and/or hot-stamped from injection-molded and vacuum-formed components. According to another embodiment, the wells each have a volume of 0.5, 1.0, 1.2, 1.5, 1.8, 2.0, or 2.5 ml. According to another embodiment, the microplate comprises 12, 24, 48, 96, or 384 wells. According to another embodiment, the microplate is a microplate according to ANSI/SLS with 96, 384, or 1,536 wells or is a deep-well plate.

### BRIEF DESCRIPTION OF THE DRAWINGS

The invention is explained in more detail below based on the accompanying drawings of an exemplary embodiment.

FIG. 1 illustrates a top perspective view of an embodiment of a microplate with a snap-fitted lid.

FIG. 2 illustrates a bottom perspective view of an embodiment of a microplate.

FIG. 3 illustrates an enlarged detail from the embodiment of FIG. 2.

FIG. 4 illustrates a top perspective view of an embodiment of a lid.

FIG. 5 illustrates a bottom perspective view of an embodiment of the lid.

FIG. 6 illustrates a partial side view and a partial longitudinal section in the longitudinal of an embodiment of the microplate with and embodiment of the snap-fitted lid.

FIG. 7 illustrates an enlarged sectional view of an embodiment of the microplate with an embodiment of the snap-fitted lid.

FIG. 8 illustrates a perspective view of an embodiment of an unlocking device.

FIG. 9 illustrates a vertical sectional view of an embodiment of the unlocking device with an embodiment of the microplate placed on and an embodiment of the lid snap-fitted thereto.

FIG. 10 illustrates a top perspective view of an embodiment of the microplate and an embodiment of the lid prior to being snap-fitted.

FIG. 11 illustrates a top perspective view or an embodiment of the microplate and an embodiment of the lid snap-fitted thereto.

FIG. 12 illustrates a top perspective view of an embodiment of the microplate and an embodiment of the lid snap-fitted thereto on an embodiment of the unlocking device during release of the snap connection.

### DETAILED DESCRIPTION

In this application, the indications “horizontal” and “vertical” as well as “top” and “bottom” relate to an arrangement of the microplate with the edge of the side wall that faces away from the first plate being on a horizontal subsurface and the lid being on the upper side of the microplate.

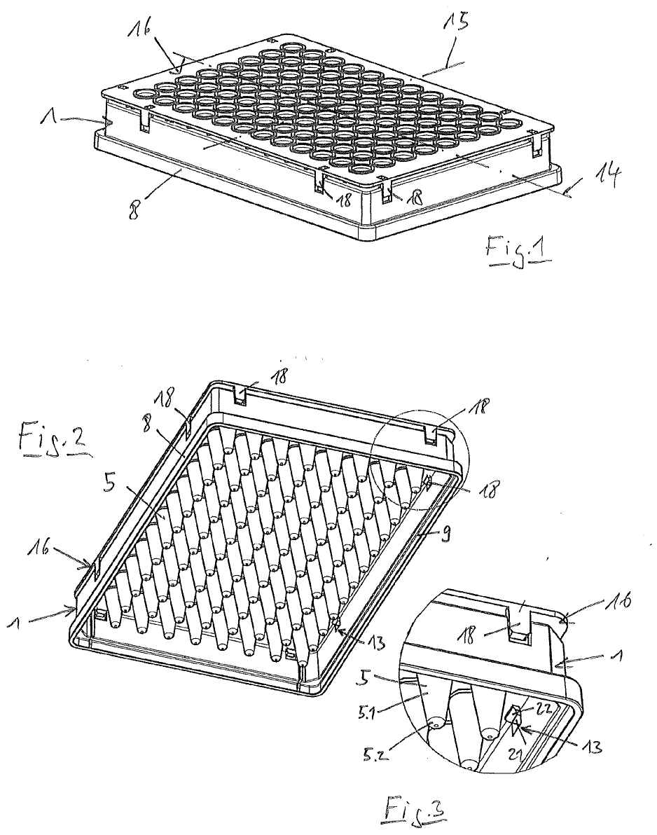

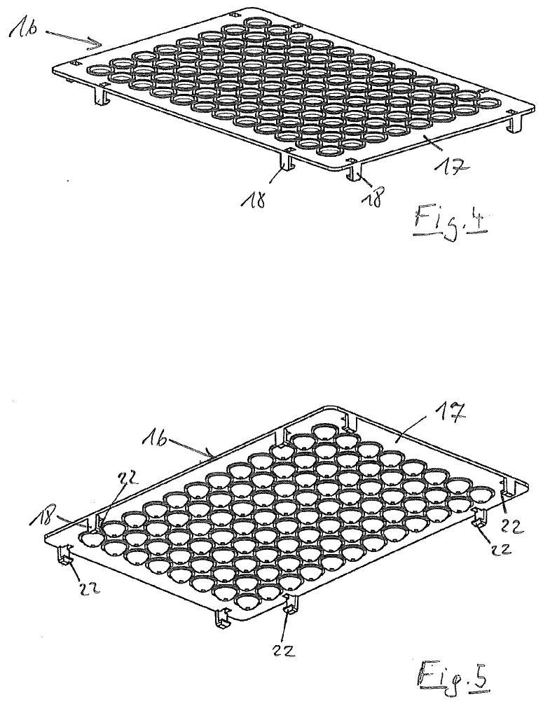

According to FIG. 1 to 3, some embodiments of a microplate 1 comprise a rectangular first plate 2 and a circumferential side wall 4 that projects downward from the outer edge 3 of the first plate 2 and that has four sides 4.1 to 4.4. The first plate 2 and the side wall 4 are also referred to as a “frame”.

Ninety-six wells 5 in eight rows and twelve columns are arranged in the first plate 2 and are connected at the top to the first plate 2, project downward from the underside of the first plate 2, and comprise an opening 6 on the upper side of the first plate 2.

Each opening 6 is surrounded by a small border 7 which projects upward from the upper side of the first plate 2.

The wells 5 have a downwardly tapering shape. They have a conical well portion 5.1 that is connected at the top to the first plate 2, with a downwardly decreasing diameter and a first downwardly curved, ball cup-shaped well base 5.2.

On the lower edge, the side wall 4 has an outwardly projecting flange 8. In the vertical section, said flange has an inverted L-profile and forms an outwardly and downwardly projecting extension 9 of the side wall 4.

On the edge of the first plate 2, the microplate 1 comprises snap openings 10. Each snap opening 10 is arranged in one part 10.1 in the lateral edge region of the first plate 2 and in another part 10.2 in the upper edge region of the side wall 4. The part 10.1 of the snap opening 10, which is formed in the lateral edge region of the first plate 2, comprises an upper boundary surface 11.1 that extends parallel to the outer edge of the first plate. Upper lateral boundary surfaces 11.2, 11.3 extend from the lateral ends of each upper boundary surface 11.1 perpendicularly thereto up to the outer edge of the first plate 2.

The other part 10.2 of the snap openings 10, which is arranged in the upper edge region of the side wall 4, comprises a lower boundary surface 12.1 that extends parallel to the upper edge of the side wall 4. Lower lateral boundary surfaces 12.2, 12.3 of the other part 10.2 of the snap openings 10 extend from the outer ends of each lower boundary surface 12.1 vertically upward up to the upper edge of the side wall 4.

The upper boundary surface 11.1 is flush with an inner side 13 of the side wall 4, which adjoins the lower boundary surface 12.1, or is offset inwards relative to said inner side 13. Two snap openings 10 are arranged in each case near a corner of the side wall 4 on each long side of the first plate 2. The snap connections 10 are arranged symmetrically with respect to a central longitudinal axis 14 of the first plate 2 on the long sides of the first plate 2.

Two snap openings 10 are arranged in each case near a corner of the side wall 4 on each short side of the first plate 2. The snap connections 10 are arranged symmetrically with respect to a central transverse axis 15 of the first plate 2 on the short sides of the first plate 2.

The microplate 1 is manufactured from a single or from multiple plastics materials, wherein the first plate 2 and the side wall 4 optionally consist of a different plastics material than the wells 5. For example, the microplate is, as a whole, manufactured from polystyrene, polycarbonate, or polypropylene or the first plate 2 and the side wall 4 consist of polycarbonate and the wells 5 consist of polypropylene, silicone, or liquid silicone rubber.

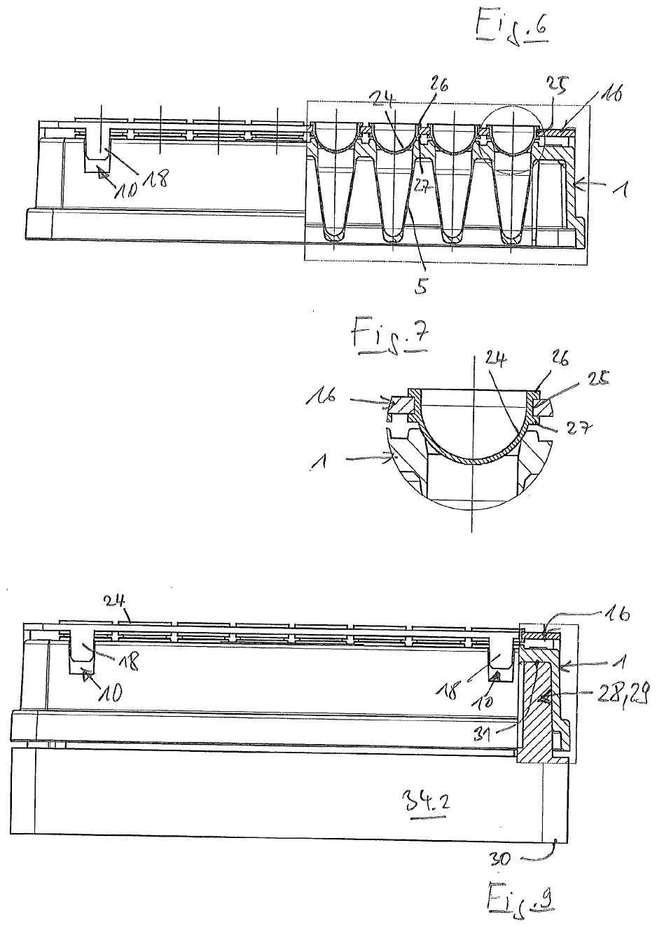

According to FIG. 1 to 3, a lid 16, which is shown in FIGS. 4 and 5, is arranged on the microplate 1. The lid 16 comprises a rectangular second plate 17 which is configured to be congruent with the first plate 2. The lid 16 comprises snap hooks 18 which project downward from the upper outer edge 19 of the second plate 17.

Each snap hook 18 comprises a vertical portion 20 which is connected at the upper end to the upper outer edge 19 of the second plate 17. Furthermore, each snap hook 18 comprises, at the lower end of the vertical portion 20, a horizontal portion 21 that projects inward, i.e. below the second plate 17. The horizontal portion 21 comprises, at the outer end on the underside, a first sliding surface 22 that is inclined with respect to the vertical.

The lid 16 has two snap hooks 18 in each case on the two long sides of the second plate 17, wherein each snap hook 18 is arranged at a corner of the second plate 17. The two snap hooks 18 on the two long sides of the second plate 17 are arranged symmetrically with respect to a central longitudinal axis 14 of the second plate 17.

The lid 16 also comprises two snap hooks 18 in each case on the two short sides of the second plate 17, each of which snap hooks is arranged near a corner of the second plate 17. The snap hooks 18 on the two shorter sides are arranged symmetrically with respect to a central transverse axis 15 of the second plate 17.

As can be seen, in particular, in FIG. 5, for the snap hooks 18 arranged symmetrically with respect to the central longitudinal axis 14 or else central transverse axis 15, the first sliding surfaces 22 are arranged in each case on inner sides of the snap hooks 18 that face one another and the same first sliding surfaces 22 are inclined upward toward one another. Consequently, the first sliding surfaces 22 on the inner sides of the snap hooks 18 facing one another enclose an acute angle with one another, the apex of which is arranged above the second plate 17.

The second plate 17 comprises a cutout 23 above the horizontal portion 22 of each snap hook 18. This simplifies removal of the lid 16 from a molding tool during injection molding. According to FIG. 4 to 6, the lid 16 comprises sealing elements 24 arranged in rows and columns on the underside of the second plate 17. Each sealing element 24 is dome-shaped (ball cup-shaped) and is held in a circular hole 25 in the second plate 17. Each sealing element 24 comprises, on the upper edge, an outwardly projecting, circumferential upper flange 26 and, therebelow and at a distance therefrom, an outwardly projecting, circumferential lower flange 27 for being held on the edge of the hole. The edge engages at a hole 25 of the second plate 17 between the upper flange 26 and the lower flange 27 of each sealing element 24, as a result of which the sealing element 24 is held on the second plate 17.

Ninety-six sealing elements 24 are arranged in the second plate 17 in eight rows and twelve columns, wherein the arrangement of the sealing elements 17 corresponds to the arrangement of the wells 5 in the microplate 1. The sealing elements 17 are designed such that each sealing element 17 can be inserted in part into a well 5 of the microplate 1, wherein the sealing element 17 sealingly abuts the upper edge of the well 5 on its inner side, as shown in FIGS. 6 and 7.

The lid 16 is manufactured by means of injection molding. The exemplary embodiment with the sealing elements 24 is manufactured in a multi-component injection-molding process, wherein the second plate 17 can be injection-molded in a first step and the sealing elements 24 can be molded onto the second plate 17 in a second step. The second plate 17 is manufactured, for example, from polystyrene, polycarbonate, or polypropylene and the sealing elements 24 from polypropylene, silicone, or liquid silicone rubber.

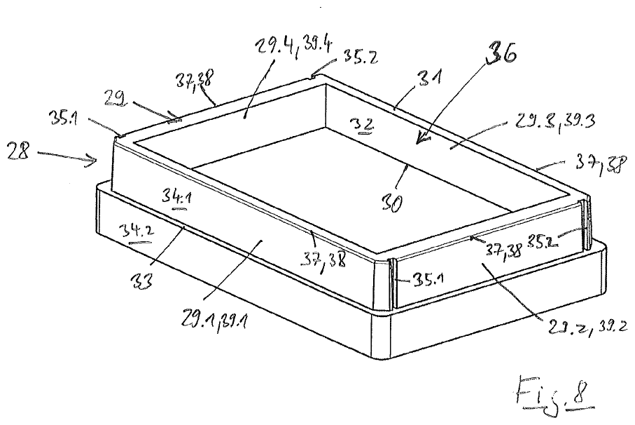

According to FIG. 8, an unlocking device 28 comprises a rectangular frame 29 which comprises four frame strips 29.1 to 29.4 that are interconnected at the corners of the frame 29. The lower edge of the frame 29 comprises a horizontal planar bearing surface 30 for placing the frame 29 on a horizontal subsurface. At the top, the frame has a horizontal planar support surface 21 for placing a microplate 1 thereon.

The frame strips in each case have a planar inner surface 32 on the inner sides. On the outer side, the frame 29 comprises a circumferential ledge 33 approximately at half height. Below the ledge 33 and above the ledge 33, the frame strips 29.1 to 29.4 each have a planar outer surface 34.1, 34.2.

Furthermore, the frame 29 comprises two vertical grooves 35.1, 35.2 in each case on the narrow frame strips 29.1, 29.2 on the outer side above the ledge, wherein each vertical groove 35.1, 35.2 is arranged next to a corner of the frame 29. The frame 29 encloses a receiving portion 36, which is delimited by the inner sides of the frame strips 29.1 to 29.4.

On the outside of the upper edge, the frame 29 comprises a circumferential chamfer 37, which forms the second sliding surfaces 38. The second sliding surfaces 38 enclose an angle with respect to the vertical. The second sliding surfaces 38 are arranged on mutually opposing frame strips 29.1 to 29.3 such that they are inclined upward toward one another. Consequently, they together enclose an acute angle with an apex above the frame 29. The frame strips 29.1 to 29.4 form support projections 39.1 to 39.4 for placing the microplate 1 thereon.

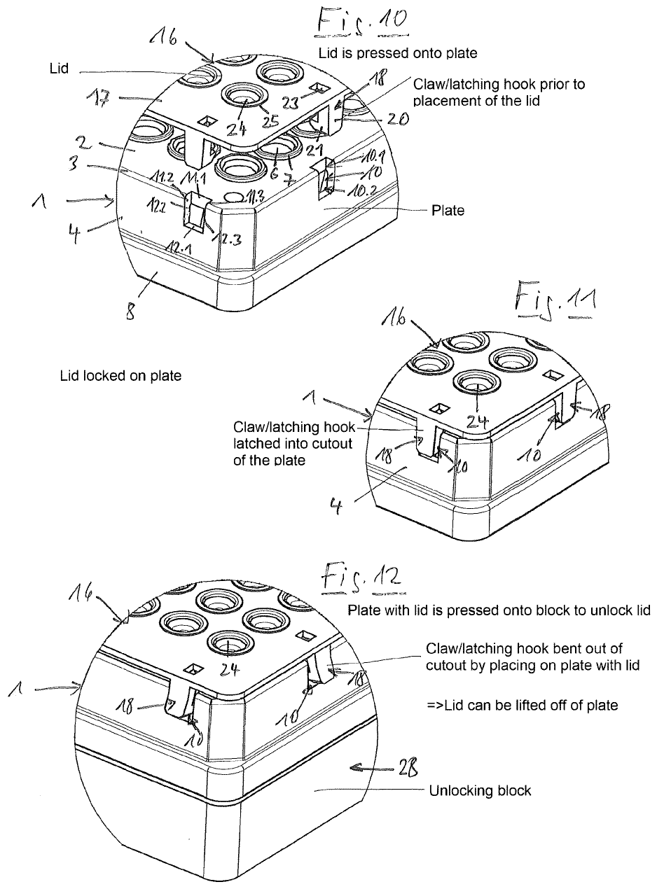

According to FIGS. 10 and 11, the lid 16 can be placed on the microplate 1, wherein the snap hooks 18 form snap connections at the snap openings 10 in which the horizontal portions 22 engage below the first plate 2 at the lateral edges. When the lid 16 has been placed on the microplate 1, the sealing elements 24 engage in a sealing manner in the openings 6 of the wells 5, as shown in FIGS. 6 and 7. In this position, the lid 16 is secured to the microplate 1 by means of the snap hooks 18.

The lid 16 is placed on the microplate 1 from above. In the process, the snap hooks 18 on the lid 16 dip into the snap openings 10 of the microplate 1. The snap hooks 18 snap in a form-fitting manner behind the edges of the snap openings 10 and hold the lid 16 securely on the microplate 1. Before the snap hooks 18 snap in, the sealing elements 24 sealingly abut the inner sides of the wells 5 in the upper region and cover said wells.

The lid 16 can be removed again by placing the microplate 1 with the lid 16 snapped on onto the unlocking device 28. The outer side and upper side of the unlocking device 28 are designed to be negative in relation to the inner side of the microplate 1. Consequently, the microplate 1 with a lid 16 snapped on thereto can be placed on the frame 29 such that the frame 29 engages from below in the circumferential side wall 4 of the microplate and the wells 5 dip into the receiving portion 36. When the microplate 1 is placed onto the unlocking device 28, vertical ribs can engage in the grooves 35.1, 35.2 of the frame 29 in order to provide structural reinforcement on the inner side of the side wall 4.

At the end of the placing movement, the first sliding surfaces 22 of the snap hooks 18 slide on the second sliding surfaces 38 of the frame 29 and, as a result, the snap hooks 18 are bent outward. As a result, the snap connections are undone, such that the lid 16 can be lifted off of and removed from the microplate 1. This is shown in FIG. 12. In the process, the lid 16 can be pushed upward from the frame 29. After releasing the snap connections, the lid 16 can be removed from the microplate 1.

The lid 16 can be placed on and snap-fitted to the microplate 1 and the snap connections released and the lid 16 removed from the microplate 1 in an automated manner, for example in a laboratory machine or dosing machine. The microplate and the lid 16 can be handled by means of grippers of an automated system (e.g. Epmotion from the applicant). Like the microplate 1, the lid 16 can be dimensionally stable and easy to handle on account of the plastics material used and, if applicable, by means of structural reinforcement (ribbing or frame).

The sealing elements 24 are preferably formed from a correspondingly soft or rather resilient material. This is, for example, a thermoplastic material, e.g. a soft PP, PE, or TPE. Each sealing element 24 can cover a well 5 on account of the flexibility of the sealing elements 24 and their arrangement in the grid of the wells 5 of the microplate 1.

The lid 16 with the sealing elements 24 may consist of multiple assembled parts or be manufactured in two-component injection molding.

### LIST OF REFERENCE SIGNS

1 Microplate
2 First plate
3 Outer edge
4 Side wall
4.1-4.4 Sides
5 Wells
5.1 Well portion
5.2 Well base
6 Opening
7 Border
8 Flange
9 Extension
10 Snap opening
10.1 Part of the snap opening
10.2 Other part of the snap opening
11.1 Upper boundary surface
11.2, 11.3 Upper lateral boundary surface
12.1 Lower boundary surface
12.2, 12.3 Lower lateral boundary surface
13 Inner side
14 Central longitudinal axis
15 Central transverse axis
16 Lid
17 Second plate
18 Snap hooks
19 Upper outer edge
20 Vertical portion
21 Horizontal portion
22 First sliding surface
23 Cutout
24 Sealing element
25 Hole
26 Upper flange
27 Lower flange
28 Unlocking device
29 Frame
29.1-29.4 Frame strip
30 Bearing surface
31 Support surface
32 Inner surface
33 Ledge
34.1-34.2 Outer surface
35.1, 35.2 Groove
36 Receiving portion
37 Chamfer
38 Second sliding surface
39.1-39.4 Support projection

1-15. (canceled)

16. A microplate set, comprising:
a microplate comprising,
a rectangular first plate comprising a upper side and an opposing underside,
a plurality of wells arranged in rows and columns, wherein each of the plurality of wells comprises a top that is connected to the rectangular first plate and projects downward from the underside of the first plate and defines an opening on the upper side of the rectangular first plate, and
snap openings on at least two mutually opposing lateral edges of the rectangular first plate;
a lid comprising,
a second plate comprising a top side and an opposing underside and configured for placement onto the upper side of the first plate to cover the opening of each of the plurality of wells and
a plurality of snap hooks projecting from the underside of the second plate on at least two mutually opposing lateral edges of the second plate, wherein the lid is configured to rest with the second plate on the upper side of the first plate to cover the opening of each of the plurality of wells, and is configured to snap into the snap openings by means of the plurality of snap hooks so as to form snap connections; and
an unlocking device configured for releasing the snap connections between the microplate and the lid, and comprising,
at least two upwardly projecting support projections and at least one receiving portion therebetween for placing a microplate with at least two mutually opposing edge regions of the first plate onto the support projections with engagement of the wells in the at least one receiving portion,
first sliding surfaces on inner sides of the plurality of snap hooks that face one another on mutually opposing edges of the first plate, and
second sliding surfaces on outer sides of the support projections that face away from one another on mutually opposing sides of the receiving portion, wherein the first sliding surfaces on inner sides of the snap hooks that face one another are inclined upward toward one another and/or the second sliding surfaces on outer sides of the support projections that face away from one another are inclined upward toward one another.

17. The set according to claim 16, wherein each of the plurality of snap openings is arranged at least in part in the two mutually opposing lateral edges of the first plate.

18. The set according to claim 16, wherein the microplate further comprises a circumferential side wall that projects downward from an outer edge of the first plate, and wherein each snap opening is formed at least in part in an upper edge region of the side wall.

19. The set according to claim 16, wherein each snap opening is arranged in one part in the lateral edge region of the first plate and in another part in an upper edge region of a side wall.

20. The set according to claim 19, wherein the part of the snap opening that is formed in the lateral edge region of the first plate comprises an upper boundary surface that extends parallel to an outer edge of the first plate and that is flush with the inner side of the side wall or is offset inward relative thereto, wherein the inner side adjoins a lower boundary surface of the part of the snap opening in the upper edge region of the side wall.

21. The set according to claim 16, wherein the microplate further comprises two snap openings defined along two long edges of the first plate, and wherein the lid further comprises two snap hooks defined along two long edges of the second plate.

22. The set according to claim 16, wherein the microplate further comprises two snap openings in defined along the two short edges of the first plate, and wherein the lid further comprises two snap hooks defined along the two short edges.

23. The set according to claim 16, wherein each snap hook comprises a vertical portion and an inwardly projecting horizontal portion at the lower end of the vertical portion.

24. The set according to claim 23, wherein the inwardly projecting horizontal portion comprises a first sliding surface at an outer end on the opposing underside.

25. The set according to claim 16, wherein the second plate comprises a cutout on the lateral edges next to the connection with the snap hook above a hook end of the snap hook.

26. The set according to claim 16, wherein at least one of the microplate and the lid comprises at least one sealing element which circumferentially seals the edge of each opening when the lid is snap-fitted to the microplate.

27. The set according to claim 16, wherein the lid comprises sealing elements arranged on the opposing underside of the second plate in rows and columns, each of which sealing elements abuts an inner edge of one of the openings when the lid has been snap-fitted to the microplate.

28. The set according to claim 16, wherein:
the first plate comprises at least one further snap opening positioned between the lateral edges;
the lid comprises at least one downwardly projecting further snap hook positioned between the two mutually opposing lateral edges of the second plate;
the at least one further snap hook is introduced into the further snap opening and is snap-fitted therebehind on the underside of the first plate when the lid, with the second plate, abuts the upper side of the first plate of the microplate; and
at least one of the unlocking device and the further snap hook comprises at least one further second sliding surface which is designed to slide on the further first sliding surface when the microplate with the lid snapped on is placed on so as to undo the snap connection.

29. The set according to claim 16, wherein:
the unlocking device further comprises a rectangular frame;
the support projections are formed by frame strips of the rectangular frame;
the receiving portion is delimited by the inner sides of the frame strips; and
a support surface is formed by upper end surfaces of the frame strips.

30. The set according to claim 16, further comprising at least one of:
the microplate is manufactured from at least one plastic material;
a rectangular frame of the microplate comprises a different plastic material than the plurality of wells;
the lid is comprised of at least one plastic materials, wherein the second plate optionally comprises a different plastics material than a sealing element;
the unlocking device comprises at least of one or more plastic materials, a metal, or a combination of one or more plastic materials and metal; and
at least one of the microplate, the lid, and the unlocking device is formed using an injection-molding process.
